(19) United States
(12) Patent Application Publication
(10) Pub. No.: US 2020/0269255 A1
(43) Pub. Date: Aug. 27, 2020

(54) AIR PURIFIER AND IMPROVEMENT OF AIR-PURIFYING PERFORMANCE

(71) Applicant: RICE EAR LTD, TAOYUAN CITY (TW)

(72) Inventors: Ying Che SUNG, TAOYUAN CITY (TW); Chung Hsin CHANG, TAOYUAN CITY (TW)

(21) Appl. No.: 16/796,540

(22) Filed: Feb. 20, 2020

(30) Foreign Application Priority Data
Feb. 26, 2019 (TW) 108106547
Feb. 26, 2019 (TW) 108202419

(51) Int. Cl. B03C 3/60; B03C 3/47; B03C 3/019; B03C 3/41; A61L 9/20

(52) U.S. Cl. CPC B03C 3/60; B03C 3/47; B03C 2201/08; B03C 3/41; A61L 9/205; B03C 3/019

(57) ABSTRACT

An air purifier includes a housing providing an air path therein, a photocatalyst module disposed in the air path, and an electrostatic dust collector disposed in the air path. The photocatalyst module includes a UV point light source emitting light along a light path, and a photocatalyst net device including at least one spatially curved member disposed in the light path, and having a configuration defined by a specific contour line of equal intensity of the emitted light. The electrostatic dust collector includes a first electrode device and a second electrode device, which have contrary electrical polarities so as to generate an electric field therebetween, and both of which have graphene on surfaces thereof.

### FIELD OF THE INVENTION

The present invention relates to an air purifier, and more particular to a photocatalyst air purifier. The present invention also relates parts of the air purifier and methods for manufacturing the parts.

### BACKGROUND OF THE INVENTION

Scientists discovered in 1972 that titanium dioxide (TiO₂) can decompose water molecules to produce hydrogen under the irradiation of ultraviolet light (wavelength is less than 380 nm). The photochemical properties of titanium dioxide make it a synonym for photocatalyst materials. Regarding “photocatalyst reaction”, as the name implies, it can use light energy to carry out a catalytic reaction on the surface of the photocatalyst material. Titanium dioxide (TiO₂) has been found to have the ability to decompose organic molecules and bacteria under the irradiation of ultraviolet light. It is convenient for people to use this photocatalyst reaction to decompose pollutants, remove odor or decompose impurities in water, and then achieve decontamination, stink removal, water purification and other effects.

In recent years, although many semiconductor materials have been found to have photocatalytic properties, subsequent studies have found that most semiconductor materials are subject to deterioration in acidic or alkaline environments, and some compounds likely result in chemical or photochemical corrosion. In contrast, titanium dioxide (TiO₂) has not only excellent photocatalytic activity, but also stable physical and chemical properties. It also has advantages of acid and alkali resistance, cheap price, easy preparation, and non-toxicity. Therefore, so far, titanium dioxide (TiO₂) has become the most widely used photocatalyst material.

Nowadays, many air purifiers have been commonly used in home appliances. Most products use filters to filter dust, and use activated carbon as deodorization means. However, a filter or an activated carbon filter is a consumable and needs to be replaced regularly. There are only a limited number of products that use photocatalyst technology to sterilize and deodorize the air passing through the air purifier. Such air purifiers generally include the following two types of designs. In one design, for example as described in Taiwanese Utility Patent No. M263951 and Chinese Patent Publication No. CN204115103U, the photocatalyst layer substantially extends along a direction perpendicular to the airflow. In the other design, the photocatalyst layer, for example as described in Taiwanese Utility Patent No. M540251, the photocatalyst layer substantially extends along a direction parallel to the airflow. Both designs aim to ensure of efficient contact between the airflow and the photocatalyst layer.

Subsequently, air purifier products that use active dust collection technology instead of passive filters for dust filtering are developed. In such an air purifier, particulate dusts in the air are charged first, and then a dust-collecting plate or dust-collecting net is changed with a polarity opposite to that of the particulate dusts. Due to the inherent attracting tendency between the positive and negative electrical charges, the particulate dusts are attracted to the dust-collecting plate. However, under the action of an electric field, the electrodes of the dust-collecting plate are continuously bombarded by the charged particles, so dusts would be accumulated on the surface of the dust-collecting plate after a certain period of time. As known, it is not easy to remove the ducts, and the electrical property and the dust-collecting efficiency would be deteriorated gradually.

Conventional washable filters are generally coated with Teflon. As known, Teflon has super hydrophobic surface characteristics, which exhibits an angle greater than 120 degrees in the water drop angle test. Unfortunately, the insulation characteristics of Teflon cannot be applied to a dust-collecting plate that needs to play the role of electrode. Furthermore, Chinese Patent Publication No. CN105251268A and Taiwanese Utility Patent No. M549778U disclose the use of pulp fibers, non-woven fabrics, and plasticized filters as the growth carriers for graphene. Graphene oxide (GO) is attached to the carrier, and then converted into graphene at a high temperature in order to perform a reduction process. However, the above-mentioned carrier materials are actually not resistant to the high temperature. Therefore, the process of reducing graphene cannot be performed at a temperature higher than 150 degrees Celsius for a long time, so it is not suitable to be used in an air purifier.

### SUMMARY OF THE INVENTION

Conventionally, a mercury lamp is used as the ultraviolet light source, which radially emits light. These years, an ultraviolet light emitting diode (UV LED) has been commonly used as the ultraviolet light source, which emits light in a manner different from the mercury lamp. The luminous pattern of the UV LED is a substantially hemispherical point light source, and the spatial distribution of the emitted light is different from the mercury lamp. Therefore, the spatial configuration of the existing photocatalyst layer would adversely affect the light-emitting efficiency of the UV LED. In addition, the conventional washable filter cannot be used as a dust-collecting plate since the dust-collecting plate according to the present invention needs to play the role of electrode.

In an aspect of the present invention, an air purifier comprises a housing providing an air path therein, a photocatalyst module disposed in the air path, and an electrostatic dust collector disposed in the air path. The photocatalyst module includes a UV point light source emitting light along a light path, and a photocatalyst net device including at least one spatially curved member disposed in the light path, and having a configuration defined by a specific contour line of equal intensity of the emitted light. The electrostatic dust collector includes a first electrode device and a second electrode device, which have contrary electrical polarities so as to generate an electric field therebetween, and both of which have graphene on surfaces thereof.

In another aspect of the present invention, a photocatalyst module for use in an air purifier, comprises at least one UV point light source for emitting UV light; and at least one arched net member aligned with the at least one UV point light source for air to be purified to pass through and for the emitted UV light of a specific intensity to reach. The intensities of the emitted UV light reaching all over the at least one arched net member are substantially equal.

In a further aspect of the present invention, a method for providing electrostatic-dust-collection function in an air purifier comprises: providing a filter carrier and applying graphene onto the filter carrier; using the filter carrier in a first electrode device, and installing the first electrode device in an air path of the air purifier; and installing a second electrode device, which has an electrical polarity contrary to the first electrode device, in the air path of the air purifier, wherein an electric field is generated between the first electrode device and the second electrode device for electrostatic dust collection.

In still another aspect of the present invention, a method for providing electrostatic-dust-collection function in an air purifier comprises: installing the first electrode device, which includes a filter carrier, in an air path of the air purifier; providing a rod array and applying graphene onto the rod array; and using the rod array in a second electrode device and installing a second electrode device, which has an electrical polarity contrary to the first electrode device, in the air path of the air purifier, wherein an electric field is generated between the first electrode device and the second electrode device for electrostatic dust collection.

### BRIEF DESCRIPTION OF THE DRAWINGS

The above contents of the present invention will become more readily apparent to those ordinarily skilled in the art after reviewing the following detailed description and accompanying drawings, in which:

FIG. 1 is a resolving diagram schematically illustrating a photocatalyst module according to an embodiment of the present invention;

FIG. 2 is a plot schematically illustrating variation of intensities with angles of emitted light;

FIG. 3A is a resolving diagram schematically illustrating a photocatalyst module according to another embodiment of the present invention;

FIG. 3B is a resolving diagram schematically illustrating a photocatalyst module according to a further embodiment of the present invention;

FIG. 4 is a flowchart schematically illustrating a method for designing an arched net member according to the present invention;

FIG. 5 is a plot schematically illustrating variations of light intensity with deflecting angles and distances from the light source on two different cross sections;

FIGS. 6A-6C are prospective, top and side views schematically illustrating a photocatalyst module similar to that shown in FIG. 3A;

FIG. 7A is a schematic diagram illustrating an electrostatic dust collector according to an embodiment of the present invention;

FIG. 7B is a schematic diagram illustrating an electrostatic dust collector according to another embodiment of the present invention; and

FIG. 8 is a schematic diagram illustrating an allocation example of an electrostatic dust collector and a photocatalyst module in a housing of an air purifier according to the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only; it is not intended to be exhaustive or to be limited to the precise form disclosed.

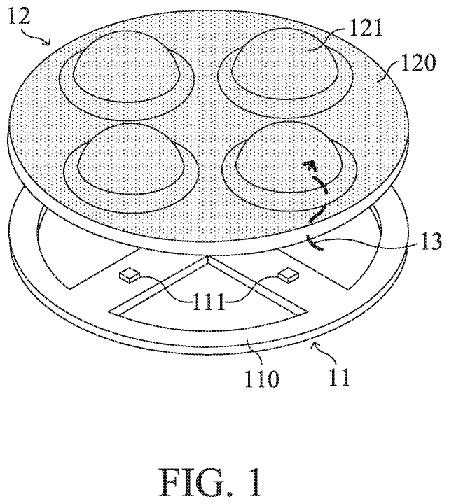

Please refer to FIG. 1, which is a schematic diagram illustrating a structure of a photocatalyst module according to an embodiment of the present invention, which can be applied to an air purifier. The photocatalyst module includes a light source device 11. The light source device 11 includes a bracket 110 and a plurality of scattered ultraviolet light sources 111, which may be implemented with ultraviolet light emitting diodes (UV LEDs). In this example, four ultraviolet light emitting diodes 111 are installed. Each of the UV LEDs 111 functions as a point light source disposed on a plane, which emits light beams distributed in a hemispherical range.

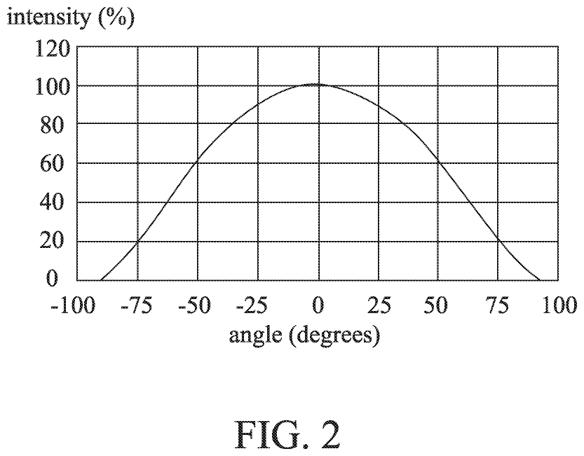

The photocatalyst module according to the present invention further includes a photocatalyst net device 12. It is understood by those skilled in the art that the emission angles of the light from the UV LEDs significantly affect the intensities of the light. The intensity of the light is highest in the direction normal to the light-emitting surface of the UV LED 111, i.e. 0 degree from the normal direction, and decreases with the increasing angle from the normal direction, as illustrated in FIG. 2, which is a plot schematically illustrating the variation of intensities with the angles of the emitted light. Therefore, the photocatalyst net device 12 is specifically configured to optimize the performance of the UV LEDs 111 in the photocatalyst module. The photocatalyst net device 12 includes a base 120 and a plurality of arched net members 121, which are disposed above the plurality of UV LED light sources 111, respectively. For example, the arched net members 121 are configured like domes or vaults. In the arched net member 121, the gap from the UV LED 111 to the roof of the dome or vault is larger in the middle and smaller in the border. Thus the intensity of light reaching different locations of the arched net member 121 could be unified. The plurality of meshes of the arched net member 121 form a plurality of air passages 13, as exemplified in FIG. 1. The arched surface, compared with a flat surface, would have a larger effective area to contact with air and conduct reaction for purification.

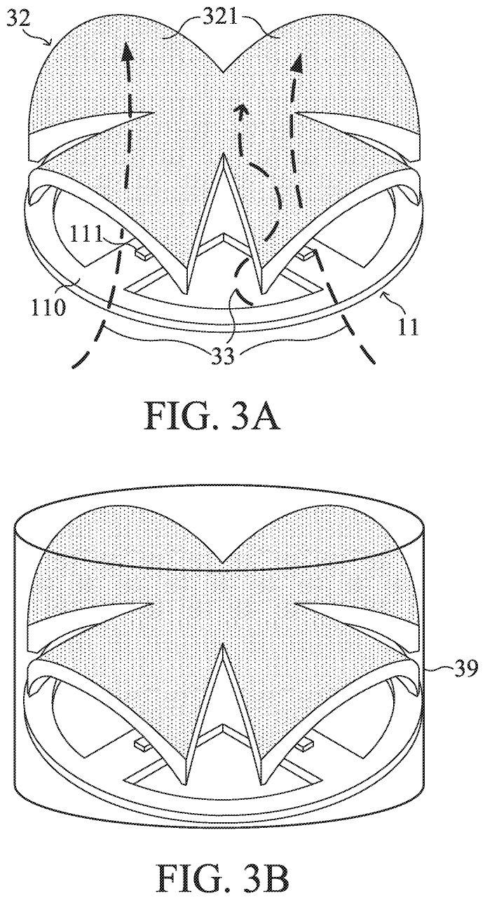

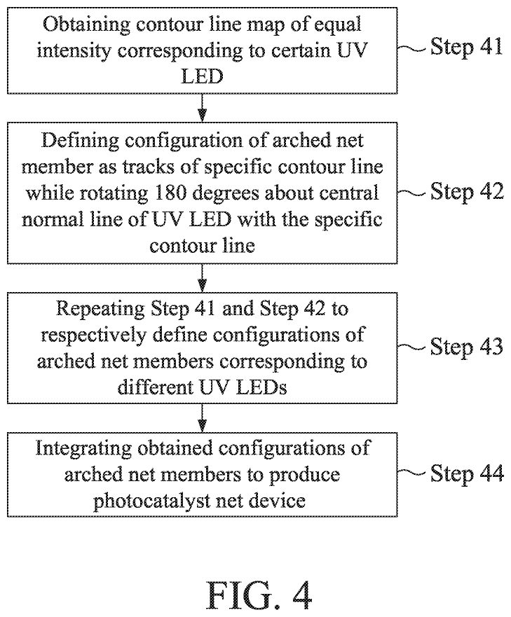

Please refer to FIG. 3A and FIG. 3B, which schematically illustrate a photocatalyst module according to another embodiment of the present invention. Similar to the embodiment illustrated in FIG. 1, the photocatalyst module includes a light source device 11 and a photocatalyst net device 32. The light source device 11 includes a bracket 110 and a plurality of scattered ultraviolet light sources 111, e.g. ultraviolet light emitting diodes (UV LEDs). The photocatalyst net device 32 includes a plurality of arched net members 321 disposed above the plurality of UV LED light sources 111, respectively. In this embodiment, the plurality of arched net members 321 are integrally formed instead of protruding from a base. The arched net members 121 and/or 321 may be made of TO₂ (titanium dioxide)-based, ZnO (zinc oxide)-based material, or any other suitable material. Likewise, in each arched net member 321, the gap from the UV LED 111 to the roof of the arched net member 321 is larger in the middle and smaller in the border. Thus the intensity of light reaching different locations of the arched net member 321 and even the photocatalyst net device 32 could be unified. The plurality of meshes of the arched net members 321 form a plurality of air passages 33, as exemplified in FIG. 3A. The arched surfaces of the photocatalyst net device 32 also provide a relatively large effective area to contact with air and conduct reaction for purification.

For enhancing optical treatment efficiency, the photocatalyst module may further includes a tubular light-reflecting chamber 39 surrounding the light source device 11 and the photocatalyst net device 32, as shown in FIG. 3B. The inner surface of the tubular light-reflecting chamber 39 is highly reflective to reflect the light emitted by the UV LED 111 inside the chamber 39 so as to increase the light amount reaching the photocatalyst net device 32, and thus improve the performance of the photocatalyst module for air purification. Similar tubular light-reflecting chamber may also be applied to the photocatalyst module illustrated in FIG. 1. Alternatively, the tubular light-reflecting chamber may be assembled to the casing of the air purifier or it may be implemented with the casing of the air purifier by having the inner surface of the casing highly reflective, for example, by coating the inner surface with a highly reflective material such as silver, aluminum, chromium, etc.

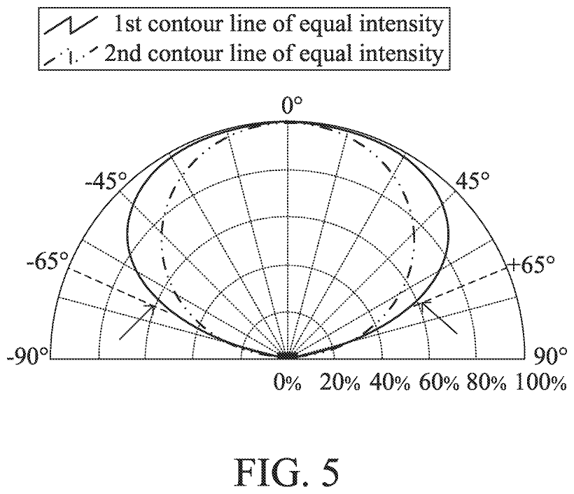

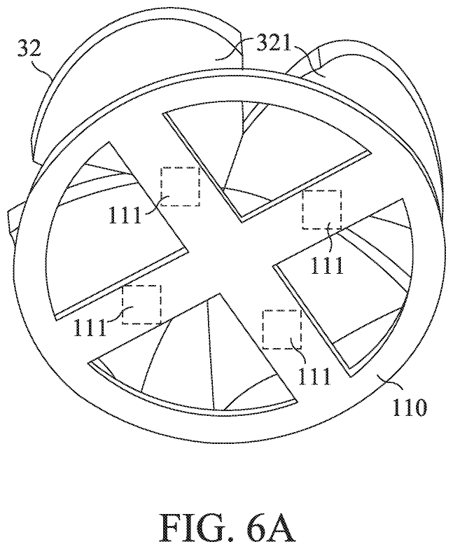

Hereinafter, creation of an arched net member according to an embodiment of the present invention is illustrated with reference to FIG. 4 and FIG. 5. FIG. 4 is a flowchart schematically illustrating the method for designing the arched net member, and FIG. 5 is a plot showing variations of light intensity with deflecting angles and distances from the light source on two different cross sections. First, light intensity distribution of a corresponding UV LED 111 and contour line maps of intensities for one or more cross sections are realized (Step 41). Each of the contour line map is created by measuring intensities of the light emitted by the UV LED 111 at different angles and different distances in the same cross section, and by connecting all the points distributed at the different angles and the different distances while having the same intensities, a contour line are formed. Since the UV LED 111 is an asymmetrically light-emitting source, the two and other contour lines taken on different cross sections would not be identical. For example, two contour lines of equal intensity, which are respectively obtained on two cross sections, are shown in FIG. 5. Once the first and second contour lines of equal intensity are realized, a modified contour line, e.g. a minimum error fitting contour line, may be created based on the two contour lines. Then the configuration of the arched net member can be defined as the tracks of the modified contour line by rotating 180 degrees about the central normal line of the light source with the modified contour line. Furthermore, it can be seen from FIG. 5 that once the deflecting angle exceeds an effective angle, e.g. positive or negative 65 degrees, the light intensity largely decays. Therefore, the portions beyond such an angle may be truncated from the modified contour line. It is to be noted that the angle “65 degrees” is only an example for illustration, and the optimal angle should be set according to practical requirements.

It is understood that the arched net member is configured based on a two-dimensional contour line in this embodiment. Alternatively, it may be configured directly based on a three-dimensional model by connecting all the points indicating identical intensities in the three-dimensional space. Then in Step 43, the operations illustrated in Step 41 and Step 42 for rendering the arched net member is repeated for each of the UV LEDs and the resulting arched net members of the equal intensity are integrated to form the photocatalyst net device (Step 44).

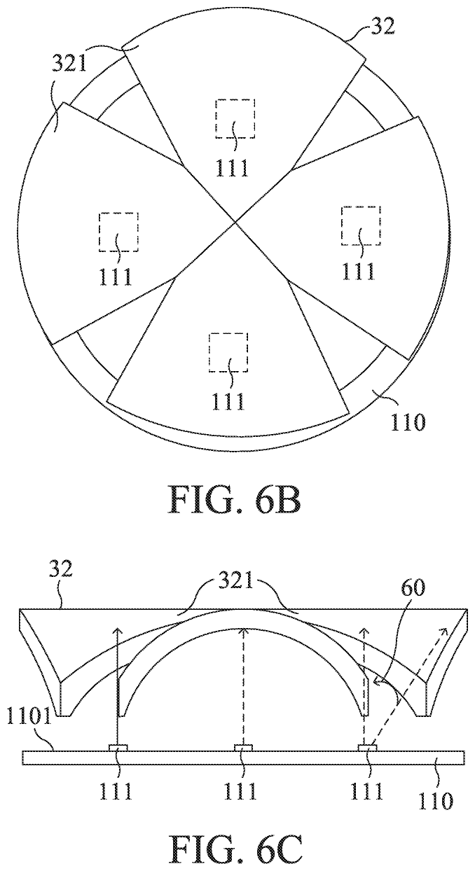

FIGS. 6A-6C are prospective, top and side views schematically illustrating an embodiment of a photocatalyst module similar to that shown in FIG. 3A, in which the relative positions of the light source device 11 and the photocatalyst module 32 can be seen. As shown, there are four UV LEDs 111 separately disposed on the upper surface 1101 of the bracket 110, and the photocatalyst module 32 are composed of four arched net members 321 aligned with the UV LEDs 111. In this embodiment, the angle 60 shown in FIG. 6C is the effective angle mentioned above, and the portions beyond the effective angle are not used to configure the arched net member. Therefore, gaps exist between adjacent arched net members. The gaps then provide air passages 33 for local turbulence as shown in FIG. 3A.

In addition to a photocatalyst module, an air purifier generally further includes an electrostatic dust collector. The electrostatic dust collector according to an embodiment of the present invention includes a first electrode device and a second electrode device, wherein the first electrode device includes a filter carrier, and the first electrode device and the second electrode device have different electrical polarities so as to generate an electric field. The filter carrier includes at least one metal mesh member, for example a metal honeycomb grid member, made of stainless steel, copper, aluminum, titanium, nickel, an alloy thereof, or any other suitable thermally resistant metal or alloy. Alternatively, the metal mesh member may also be configured as single or multiple layers or a metal foam. According to the present invention, graphene is further applied to the metal mesh member by spraying, coating, dipping, or any other suitable means. As known to those skilled in the art, graphene has good electric conductivity, better than copper and silver, so it is suitable to be used in the electrode device. Graphene is highly hydrophobic, similar to Teflon, so it is easy to clean and repetitively used. Furthermore, since graphene has extremely high specific surface area, i.e. total surface area of a material per unit of mass or solid or bulk volume, it is advantageous in effectively adsorbing large amount of dust. Therefore, the metal mesh member with graphene can improve air-purifying performance, and the quality of graphene significantly affects the air-purifying performance.

According to an embodiment of the present invention, a graphene oxide (GO) solution having a GO concentration ranged between 0.1% and 5% and a number of GO layers ranged between 1 and 20 is prepared. By dipping the filter carrier into the GO solution or spraying the GO solution onto the filter carrier, graphene oxide is attached onto the surface of the filter carrier, and then a baking process at a temperature over 150 degrees Celsius is performed to conduct a reduction reaction, thereby providing a thin graphene stack on the surface of the filter carrier.

In another embodiment, graphene is mixed into a colloid to form a mixture having graphene contents of 0.01%-2%. The mixture is then applied onto the surface of the filter carrier to provide good hydrophobicity. Preferably, the number of graphene atom layers is under 20. The resulting filter carrier has good electrical conductivity and is suitable for dust collection. In addition, the specific surface area is large, for example greater than 100 square meters/g), and thus the probability of collision of air particles in the air flow, as well as the probability of retaining/filtering collision particles, are increased. Furthermore, a low number of graphene atom layers has extremely low surface energy, so external charged particles would only temporarily stay in graphene gaps in a loose manner. The particles can be easily washed away to restore the original state of the filter carrier.

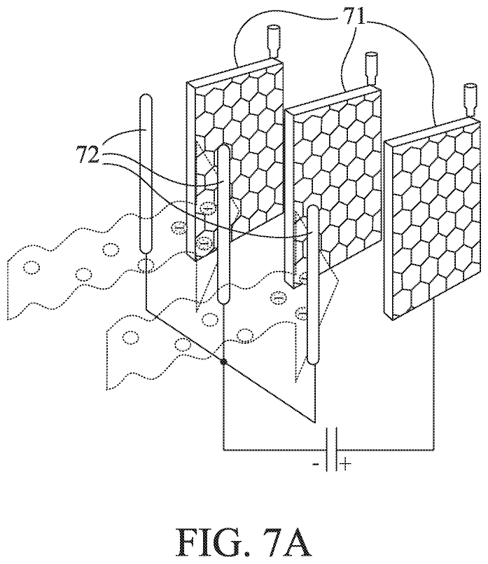

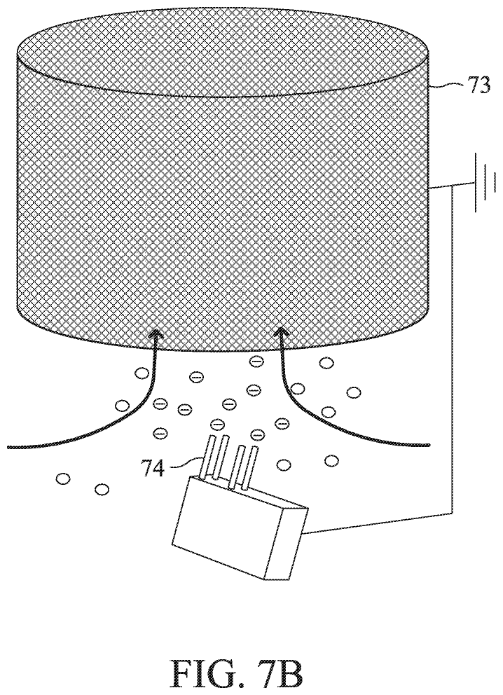

Please refer to FIG. 7A, in which an electrostatic dust collector according to an embodiment of the present invention is schematically illustrated. As shown, the electrostatic dust collector includes a first electrode device 71 and a second electrode device 72. The first electrode device 71 includes a filter carrier, which is composed of one or more dust-collecting plates, and each of the plates has a honeycomb grid configuration with graphene coating prepared as described above. The second electrode device 72 includes a discharging device, which is rod-shaped, for charging particulate dust in the air. The particulate dust thus carries negative charges. The electrical polarities of the filter carrier and the discharging device are contrary so that the particulate dust can be attracted and collected by the dust-collecting plates.

Please refer to FIG. 7B, in which an electrostatic dust collector according to another embodiment of the present invention is schematically illustrated. As shown, the electrostatic dust collector includes a first electrode device 73 and a second electrode device 74. The first electrode device 73 includes a filter carrier, which is configured as a hollow cylinder with open bottom and coated with graphene. The second electrode device 74 includes a discharging device, which is rod-shaped, for charging particulate dust in the air. The particulate dust thus carries negative charges. The electrical polarities of the filter carrier and the discharging device are contrary so that the particulate dust can be attracted and collected by the dust-collecting cylinder.

In the discharging device included in the second electrode device 72 or 74, a transformer is used to generate high AC voltage, which is converted into a high negative DC voltage by a rectifier circuit. Then the high AC voltage is applied to the negative electrode of the discharging device 72 or 74, thereby releasing electrons and generating negative charges. The lowest voltage that can cause ion discharge is defined as an initial discharge voltage, and the initial discharge voltage varies with material of the negative electrode and tip radius of curvature. Please refer to the data in the table below.

| | 0.5 mm | | 0.1 mm | 0.05 mm | 0.01 mm | <0.01 mm |
|---|---|---|---|---|---|---|
| copper | 10.2 | kV | 7.4 kV | 6.9 kV | 5.3 kV | 2.2 kV |
| silver | 9.1 | kV | 6.8 kV | 5.8 kV | 4.7 kV | 4.9 kV |
| tungsten | 8.8 | kV | 7.3 kV | 5.7 kV | 4.0 kV | 4.5 kV |
| graphite | 10.9 | kV | 7.7 kV | 7.9 kV | 7.5 kV | 5.1 kV |
| carbon | 8.1 | kV | 7.4 kV | 6.2 kV | 6.6 kV | 6.6 kV |
| fiber | | | | | | |

For the same material, the smaller the tip radius, the smaller the initial discharge voltage, and the more the discharged ions. Commercially, the discharging electrodes are implemented with copper needles or carbon brushes. The radius of the tip of a common carbon brush is about 0.015 mm, and the total number of carbon brush tips is about 1 k-10 k. The air-purifying performance of the conventional means, however, is not satisfactory.

According to the present invention, the discharging device included in the second electrode device 72 or 74 is composed of rod array formed of zinc oxide (ZnO). The zinc oxide rods are of a nanoscale so that the amount of discharged ions can be largely increased by using the zinc oxide rods for tip discharging. The ZnO rod array may be produced by, for example, hydrothermal process, sputtering, low pressure chemical vapor deposition (LPCVD), and any other suitable process. It is to be noted that when discharging is conducted in the atmospheric environment, the strong discharge effect will cause the ZnO rod array suffering from discharge corrosion and thus damage of structure. Furthermore, the discharge effect might be weakened or even terminated. Therefore, in a further embodiment, graphene is applied to the surfaces of the ZnO rods for protection. For example, apply graphene oxide (GO) onto the surfaces of the ZnO rods by way of coating, dipping, spraying, or any other suitable technique. Conduct a reduction reaction of graphene oxide into graphene at a temperature ranged between 100 and 450 degrees Celsius. The reduced graphene contents would be 0.1 wt % to 10 wt % and the graphene stack would include 1 to 20 layers of graphene atoms. Furthermore, the high electrical conductivity of graphene facilitates dispersion of the external high-voltage power on the surface of the nanoscale rods to avoid unevenly discharging. Furthermore, the ZnO rods with graphene is capable of preventing electric corrosion, which results from contact of the ZnO rods with water molecules in the air during the high-voltage discharge process. According to the present invention, in addition to zinc oxide (ZnO), there are still other materials, which are suitable to form the discharging device to improve charging efficiency. Examples include silicon and silver. Nanoscale silicon rods or nanoscale silver rods may be formed by etching silicon or silver raw material into the desired structure.

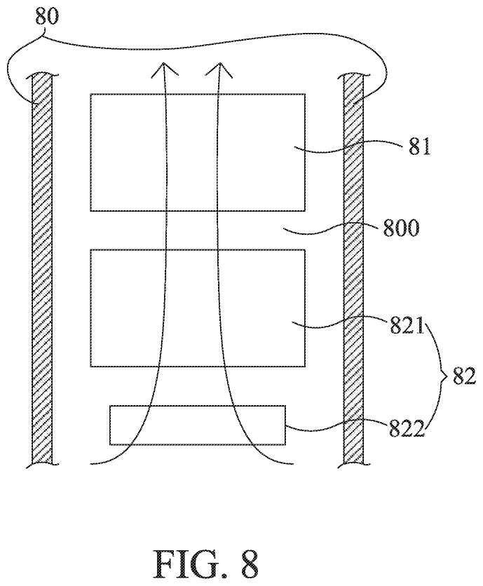

Please refer to FIG. 8, which schematically exemplifies allocation of an electrostatic dust collector 82 and a photocatalyst module 81 in a housing 80 of an air purifier according to the present invention. Inside the housing 80, air to be purified transmits through the inner space of the air purifier along the path 800, where the photocatalyst module 81 and the electrostatic dust collector 82 are disposed. The photocatalyst module 81 and the electrostatic dust collector 82 may be implemented with the ones illustrated in the above embodiments. The electrostatic dust collector 82 includes a first electrode device 821 and a second electrode device 822, both of which having a proper graphene stack thereon. The first electrode device 821 and the second electrode device 822 have contrary electrical polarities and form an electric field therebetween. As described above, these designs result in good photocatalytic reaction efficiency, easy cleaning of the electrostatic dust filter, and the durability of the discharge electrode, as well as improvement of discharging efficiency.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. An air purifier, comprising:
a housing providing an air path therein;
a photocatalyst module disposed in the air path, and including:
a UV point light source emitting light along a light path; and
a photocatalyst net device including at least one spatially curved member disposed in the light path, and having a configuration defined by a specific contour line of equal intensity of the emitted light; and
an electrostatic dust collector disposed in the air path, and including a first electrode device and a second electrode device, which have contrary electrical polarities so as to generate an electric field therebetween, and both of which have graphene on surfaces thereof.

2. The air purifier according to claim 1, wherein the UV point light source is a UV LED, and the spatially curved is an arched net member made of a TiO₂-based or ZnO-based material.

3. The air purifier according to claim 1, wherein the first electrode device includes a metal filter carrier having thereon a graphene stack formed of 1-20 layers of graphene atoms.

4. The air purifier according to claim 1, wherein the second electrode device includes a discharging device configured as a rod array and having thereon a graphene stack formed of 1-20 layers of graphene atoms.

5. A photocatalyst module for use in an air purifier, comprising:
at least one UV point light source for emitting UV light; and
at least one arched net member aligned with the at least one UV point light source for air to be purified to pass through and for the emitted UV light of a specific intensity to reach,
wherein the intensities of the emitted UV light reaching all over the at least one arched net member are substantially equal.

6. The photocatalyst module according to claim 5, wherein the at least one UV point light source is installed on a bracket.

7. The photocatalyst module according to claim 5, wherein the arched net member is made of a TiO₂-based or ZnO-based material.

8. The photocatalyst module according to claim 5, wherein the photocatalyst module includes a plurality of the arched net members, and a gap exists between adjacent ones of the arched net members for air to be purified to pass through so as to result in local turbulence.

9. A method for providing electrostatic-dust-collection function in an air purifier, comprising:
providing a filter carrier and applying graphene onto the filter carrier;
using the filter carrier in a first electrode device, and installing the first electrode device in an air path of the air purifier; and
installing a second electrode device, which has an electrical polarity contrary to the first electrode device, in the air path of the air purifier, wherein an electric field is generated between the first electrode device and the second electrode device for electrostatic dust collection.

10. The method according to claim 9, wherein the filter carrier is a metal filter carrier, and graphene is applied onto the metal filter carrier by:
applying a graphene oxide (GO) solution onto metal filter carrier; and
heating the metal filter carrier with the GO solution to reduce graphene oxide into graphene.

11. The method according to claim 10, wherein the GO solution has a GO concentration of 0.1%-5% and is applied to the metal filter carrier by dipping, spraying or coating.

12. The method according to claim 10, wherein the metal filter carrier with the GO solution is heated at a temperature higher than 150 degrees Celsius.

13. The method according to claim 9, wherein graphene is applied onto the filter carrier as a graphene stack of 1-20 layers of graphene atoms.

14. The method according to claim 10, wherein the second electrode device includes a rod array for tip discharging, and the method further comprises applying graphene onto the rod array.

15. The method according to claim 14, wherein the rod array is made of zinc oxide.

16. The method according to claim 14, wherein graphene is applied onto the rod array by:
applying a graphene oxide (GO) solution onto the rod array; and
heating the rod array with the GO solution to reduce graphene oxide into graphene.

17. The method according to claim 16, wherein the GO solution has a GO concentration of 0.1%-5% and is applied to the rod array by dipping, spraying or coating.

18. The method according to claim 16, wherein the rod array with the GO solution is heated at a temperature ranged between 100 and 450 degrees Celsius.

19. The method according to claim 14, wherein graphene is applied onto the rod array as a graphene stack of 1-20 layers of graphene atoms.

20. A method for providing electrostatic-dust-collection function in an air purifier, comprising:
installing the first electrode device, which includes a filter carrier, in an air path of the air purifier;
providing a rod array and applying graphene onto the rod array; and
using the rod array in a second electrode device and installing a second electrode device, which has an electrical polarity contrary to the first electrode device, in the air path of the air purifier, wherein an electric field is generated between the first electrode device and the second electrode device for electrostatic dust collection.
